(19) United States
(12) Patent Application Publication
(10) Pub. No.: US 2023/0276831 A1
(43) Pub. Date: Sep. 7, 2023

(54) Food/Beverage Article

(71) Applicant: Kikkoman Corporation, Chiba (JP)

(72) Inventors: Eiji Shimabe, Noda-shi, Chiba (JP); Riichiro Uchida, Noda-shi, Chiba (JP); Sachiko Kobayashi, Noda-shi, Chiba (JP); Kazuya Marushima, Noda-shi, Chiba (JP); Yoshikazu Endo, Noda-shi, Chiba (JP); Takuya Ohira, Noda-shi, Chiba (JP)

(73) Assignee: Kikkoman Corporation, Chiba (JP)

(21) Appl. No.: 18/019,872

(22) PCT Filed: Aug. 2, 2021
(86) PCT No.: PCT/JP2021/028606

§ 371 Date: Feb. 6, 2023

(30) Foreign Application Priority Data
Aug. 7, 2020 (JP) 2020-135109

(51) Int. Cl. A23L 3/3544; A23L 27/50; A23L 23/00

(52) U.S. Cl. CPC A23L 3/3544; A23L 27/50; A23L 23/00

(57) ABSTRACT

A food/beverage article, comprising:
one or more compounds represented by formula (I):

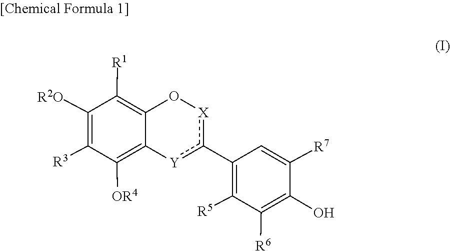

wherein, R¹ and R³ each independently represent a hydrogen atom or a C₂₋₆ alkenyl group, R² and R⁴ each independently represent a hydrogen atom or a C₁₋₄ alkyl group, R⁵ and R⁷ each independently represent a hydrogen atom or a hydroxyl group, R⁶ represents a hydrogen atom or a C₂₋₆ alkenyl group, X represents a direct bond, —CH₂—, —CH═, or —C(═O)—, and Y represents —CH₂—, —CH═, or —C(═O)—, wherein the total of the concentrations of the compounds is 1 ppm or more.

The present invention relates to a food/beverage article containing an oxygen-containing heterocyclic compound.

### BACKGROUND ART

Low fungus-resistant food/beverage articles such as low-salt soy sauce and ready-to-use Japanese seasoned soup stock containing a small amount of salt have been distributed due to an increase in the health orientation of consumers and the like in recent years. If such low fungus-resistant food/beverage articles are contaminated with lactic acid bacteria, the food/beverage articles are deteriorated, resulting in, for example, pH change due to the generation of lactic acid or the decarboxylation of amino acids, and the deformation of the containers due to the generation of carbonic acid gas, and thus the qualities of products are markedly spoiled disadvantageously (for example, Non Patent Literature 1). Also, if low fungus-resistant food/beverage articles are contaminated with flat sour bacteria, the contents are acidified due to acid production and the qualities of products are markedly spoiled disadvantageously (for example, Non Patent Literature 2).

### CITATION LIST

### Non Patent Literature

### Non Patent Literature 1

JOURNAL OF THE BREWING SOCIETY OF JAPAN, 2008, Vol. 103, No. 2, p. 94-99

### Non Patent Literature 2

B. coagulans, edited by Tateo Fujii, Fundamentals of Food Microbiology, KODANSHA LTD., 2015, p. 15

### SUMMARY OF INVENTION

### Technical Problem

Examples of means for suppressing the deterioration of food/beverage articles due to contamination with lactic acid bacteria and/or flat sour bacteria include synthetic preservative addition, an increase in the amount of salt and/or alcohol, and acidification by reducing the pH. It cannot, however, be said that any method described above is desirable in view of health orientation and the savor of food.

An object of the present invention is to provide a food/beverage article that is unlikely to be deteriorated due to lactic acid bacteria and/or flat sour bacteria contamination without depending on these methods.

### Solution to Problem

As a result of earnest studies to solve the above-mentioned problem, the present inventors have consequently found that an oxygen-containing heterocyclic compound such as licoricidin represented by the following formula (I) exhibits antimicrobial activity to lactic acid bacteria and/or flat sour bacteria, and completed the present invention based on the finding.

Specifically, the present invention provides a food/beverage article comprising: one or more compounds represented by the following formula (I):

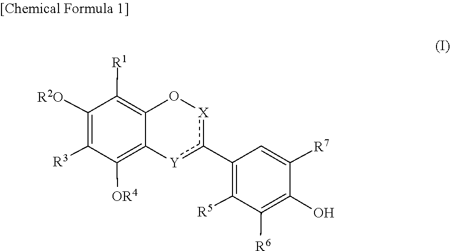

wherein, R¹ and R³ each independently represent a hydrogen atom or a C₂₋₆ alkenyl group, R² and R⁴ each independently represent a hydrogen atom or a C₁₋₄ alkyl group, R⁵ and R⁷ each independently represent a hydrogen atom or a hydroxyl group, R⁶ represents a hydrogen atom or a C₂₋₆ alkenyl group, X represents a direct bond, —CH₂—, —CH═, or —C(═O)—, Y represents —CH₂—, —CH═, or —C(═O)—, and a bond represented by the following formula:

[Chemical Formula 2]

[symbol]

represents a single bond or a double bond (hereinafter also referred to as a “compound (I)”), wherein the total of the concentrations of the compounds is 1 ppm or more. Since the food/beverage article of the present invention contains the compound (I) at 1 ppm or more, the food/beverage article exhibits antimicrobial activity to lactic acid bacteria and/or flat sour bacteria, and is unlikely to be deteriorated due to lactic acid bacteria and/or flat sour bacteria contamination.

It is preferable that the compound (I) be at least one selected from the group consisting of licoricidin, gancaonin I, 8-(γ,γ-dimethylallyl)-wighteone, glycycoumarin, glyasperin C, glycyrin, isoangustone A, and licoarylcoumarin.

The food/beverage article of the present invention may be liquid seasoning or food. The above-mentioned liquid seasoning may be soy sauce, soup stock, Japanese seasoned soup stock, sauce, dressing, or cooking vinegar, and the above-mentioned food may be lightly-pickled vegetables.

The present invention provides an antimicrobial agent to lactic acid bacteria and/or flat sour bacteria, comprising: the compound (I) as an active ingredient.

The present invention provides a method for suppressing the proliferation of lactic acid bacteria and/or flat sour bacteria in the food/beverage article, comprising: adjusting the total of the concentrations of the compounds (I) in the food/beverage article to 1 ppm or more.

### Advantageous Effects of Invention

According to the present invention, a food/beverage article is unlikely to be deteriorated due to lactic acid bacteria and/or flat sour bacteria contamination can be provided. According to the present invention, a novel antimicrobial agent to lactic acid bacteria and/or flat sour bacteria can be provided. According to the present invention, a method for suppressing the proliferation of lactic acid bacteria and/or flat sour bacteria in the food/beverage article can be provided.

### DESCRIPTION OF EMBODIMENTS

Hereinafter, preferred embodiments of the present invention will be described in detail. However, the present invention is not limited to the following embodiments.

A food/beverage article according to the present embodiment contains a compound represented by the following formula (I) at 1 ppm or more:

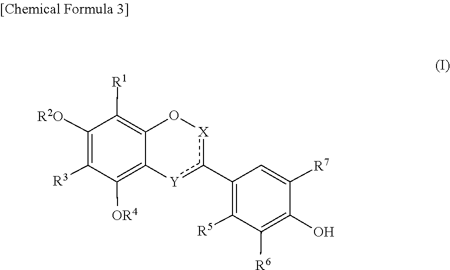

wherein, R¹ and R³ each independently represent a hydrogen atom or a C₃₋₆ alkenyl group, R² and R⁴ each independently represent a hydrogen atom or a C₁₋₄ alkyl group, R⁵ and R⁷ each independently represent a hydrogen atom or a hydroxyl group, R⁶ represents a hydrogen atom or a C₂₋₆ alkenyl group, X represents a direct bond, —CH₂—, —CH═, or —C(═O)—, Y represents —CH₂—, —CH═, or —C(═O)—, and a bond represented by the following formula:

[Chemical Formula 4]

[symbol]

represents a single bond or a double bond.

The “C₁₋₄ alkyl group” used herein means a linear or branched alkyl group having 1 to 4 carbon atoms. Examples of the C₁₋₄ alkyl group include a methyl group, an ethyl group, a propyl group, an isopropyl group, a butyl group, an isobutyl group, a sec-butyl group, and a tert-butyl group.

The “C₂₋₆ alkenyl group” used herein means a linear or branched alkenyl group having 2 to 6 carbon atoms. Examples of the C₂₋₆ alkenyl group include a vinyl group, a propen-1-yl group, a propen-2-yl group, a 1-butenyl group, a 2-butenyl group, a 3-butenyl group, a 1-methyl-1-propenyl group, a 2-methyl-1-propenyl group, a 1-pentenyl group, a 2-pentenyl group, a 3-pentenyl group, a 4-pentenyl group, a 5-pentenyl group, a 1-methyl-1-butenyl group, a 2-methyl-1-butenyl group, a 3-methyl-1-butenyl group, a 4-methyl-1-butenyl group, a 1-methyl-2-butenyl group, a 2-methyl-2-butenyl group, a 3-methyl-2-butenyl group, a 4-methyl-2-butenyl group, a 1-methyl-3-butenyl group, a 2-methyl-3-butenyl group, a 3-methyl-3-butenyl group, a 4-methyl-3-butenyl group, a 1,2-dimethyl-1-propenyl group, a 1,1-dimethyl-2-propenyl group, a 1-hexenyl group, a 2-hexenyl group, a 3-hexenyl group, a 4-hexenyl group, a 5-hexenyl group, and a 6-hexenyl group.

In above-mentioned formula (I), R¹ is preferably a hydrogen atom, a 3-methyl-2-butenyl group, or a 1,1-dimethyl-2-propenyl group.

In above-mentioned formula (I), R² is preferably a hydrogen atom or a methyl group.

In above-mentioned formula (I), R³ is preferably a hydrogen atom or a 3-methyl-2-butenyl group.

In above-mentioned formula (I), R⁴ is preferably a hydrogen atom or a methyl group.

In above-mentioned formula (I), R⁶ is preferably a hydrogen atom or a 3-methyl-2-butenyl group.

The compound (I) may have isomers such as stereoisomers and tautomers. Those isomers are also included in the scope of the present invention.

Specific examples of the compound (I) include licoricidin, gancaonin I, 8-(γ,γ-dimethylallyl)-wighteone, glycycoumarin, glyasperin C, glycyrin, isoangustone A, and licoarylcoumarin.

Licoricidin is also called 4-[(R)-7-hydroxy-5-methoxy-6-(3-methyl-2-butenyl)chroman-3-yl]-2-(3-methyl-2-butenyl)-1,3-benzenediol, and is a known compound represented by the following formula:

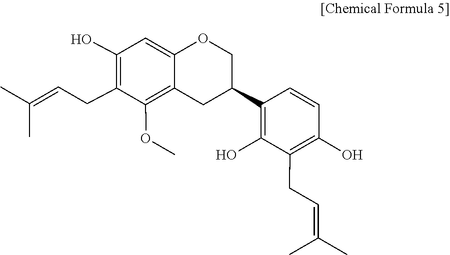 [SMILES: COc1c(CC=C(C)C)c(O)cc2c1C[C@H](c1ccc(O)c(CC=C(C)C)c1O)CO2]

Gancaonin I is also called 5-(3-methyl-2-butenyl)-2-(2,4-dihydroxyphenyl)-4,6-dimethoxybenzofuran, and is a known compound represented by the following formula:

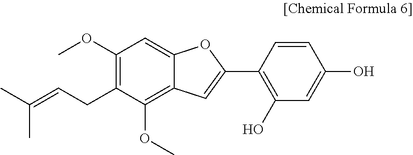 [SMILES: COc1cc2oc(-c3ccc(O)cc3O)cc2c(OC)c1CC=C(C)C]

8-(7,7-Dimethylallyl)-wighteone is also called 6,8-bis(3-methyl-2-butenyl)-3-(4-hydroxyphenyl)-5,7-dihydroxy-4H-1-benzopyran-4-one, and is a known compound represented by the following formula:

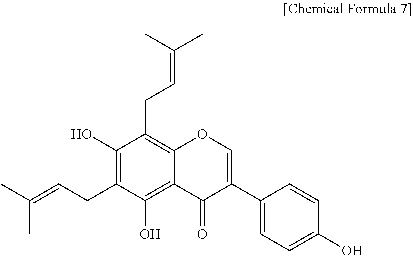 [SMILES: CC(C)=CCc1c(O)c(CC=C(C)C)c2occ(-c3ccc(O)cc3)c(=O)c2c1O]

Glycycoumarin is also called 3-(2,4-dihydroxyphenyl)-7-hydroxy-5-methoxy-6-(3-methyl-2-butenyl)-2H-1-benzopyran-2-one, and is a known compound represented by the following formula:

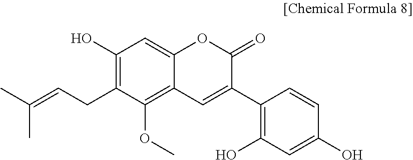 [SMILES: COc1c(CC=C(C)C)c(O)cc2oc(=O)c(-c3ccc(O)cc3O)cc12]

Glyasperin C is also called (3R)-3β(2,4-dihydroxyphenyl)-5-methoxy-6-(3-methyl-2-butenyl)-3,4-dihydro-2H-1-benzopyran-7-ol, and is a known compound the following formula:

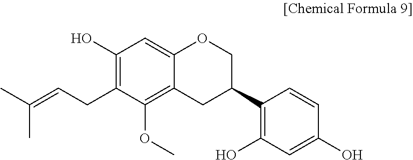 [SMILES: COc1c(CC=C(C)C)c(O)cc2c1C[C@H](c1ccc(O)cc1O)CO2]

Glycyrin is also called 3-(2,4-dihydroxyphenyl)-5,7-dimethoxy-6-(3-methyl-2-butenyl)-2H-1-benzopyran-2-one, and is a known compound represented by the following formula:

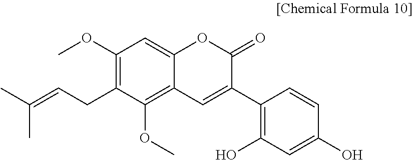 [SMILES: COc1cc2oc(=O)c(-c3ccc(O)cc3O)cc2c(OC)c1CC=C(C)C]

Isoangustone A is also called 3-[3,4-dihydroxy-5-(3-methyl-2-butenyl)phenyl]-5,7-dihydroxy-6-(3-methyl-2-butenyl)-4H-chromen-4-one, and is a known compound represented by the following formula:

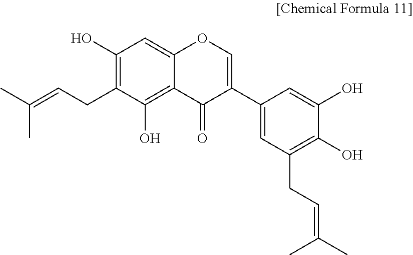 [SMILES: CC(C)=CCc1cc(-c2coc3cc(O)c(CC=C(C)C)c(O)c3c2=O)cc(O)c1O]

Licoarylcoumarin is also called 3-(2,4-dihydroxyphenyl)-7-hydroxy-5-methoxy-8-(2-methyl-3-butene-2-yl)coumarin, and is a known compound represented by the following formula:

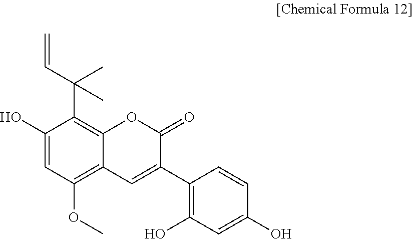 [SMILES: C=CC(C)(C)c1c(O)cc(OC)c2cc(-c3ccc(O)cc3O)c(=O)oc12]

The compound (I) may be synthesized by a method known to those skilled in the art, or may be a commercially available product. Licoricidin, gancaonin I, 8-(γ,γ-dimethylallyl)-wighteone, glycycoumarin, glyasperin C, glycyrin, isoangustone A, and licoarylcoumarin are known to be compounds contained in licorice oily extract. Accordingly, in the food/beverage article according to the present embodiment, licorice oily extract containing the compound (I) can also be used as it is, or the compound (I) contained in licorice oily extract can be isolated therefrom before use.

The food/beverage article according to the present embodiment may contain only one compound (I), or may contain two or more compounds (I). When the food/beverage article according to the present embodiment contains two or more compounds (I), the concentration of the compounds (I) means the total concentration of the concentrations of the compounds.

In the food/beverage article according to the present embodiment, the concentration of the compound (I) is 1 ppm or more, in terms of the lower limit. This enables suppression of the deterioration of the food/beverage article due to lactic acid bacteria and/or flat sour bacteria contamination. The concentration of the compound (I) can be suitably set to the range of 1 ppm or more according to the use of the food/beverage article, the salt concentration, the alcohol concentration, the pH, and the like, and the concentration of the compound (I) may be, for example, 5 ppm or more, may be 10 ppm or more, may be 25 ppm or more, or may be 50 ppm or more in view of further enhancing the antimicrobial activity to lactic acid bacteria and/or flat sour bacteria.

In the food/beverage article according to the present embodiment, the upper limit of the concentration of the compound (I) is not particularly limited, and the concentration of the compound (I) may be, for example, 10000 ppm or less in view of dissolving the compound (I) in the target food/beverage article adequately.

For example, the concentration of the compound (I) can be measured by liquid chromatography and tandem mass spectrometry (LC/MS/MS).

The food/beverage article according to the present embodiment may be any food/beverage article without particular limitation, as long as it is a food/beverage article containing the compound (I) at 1 ppm or more. Examples of the food/beverage article according to the present embodiment include liquid seasonings such as soy sauce, soup stock, Japanese seasoned soup stock, sauce, soup, Worcester sauce, and dressing; semisolid seasonings such as miso and mayonnaise; pickled Japanese plums; pickles; and delicatessen. It is preferable that the food/beverage article according to the present embodiment be liquid seasoning or food, since the deterioration due to lactic acid bacteria and/or flat sour bacteria contamination can be further suppressed. Soy sauce, soup stock, Japanese seasoned soup stock, sauce, dressing, or cooking vinegar is preferable among the liquid seasonings. Lightly-pickled vegetables are preferable among the foods.

Although the salt concentration of the food/beverage article according to the present embodiment is not particularly limited, the salt concentration may be, for example, 12% (w/v) or less, 8% (w/v) or less, 4% (w/v) or less, or 0% (w/v) (unsalted), in view of enhancing the antimicrobial activity to lactic acid bacteria and/or flat sour bacteria without increasing the amount of salt.

For example, the salt concentration can be measured by a known method such as potentiometric titration, the Mohr method, or atomic absorption spectrophotometry.

Although the alcohol concentration of the food/beverage article according to the present embodiment is not particularly limited, the alcohol concentration may be, for example, 10% (v/v) or less, 5% (v/v) or less, or 0% (v/v), in view of enhancing the antimicrobial activity to lactic acid bacteria and/or flat sour bacteria without increasing the amount of alcohol.

For example, the alcohol concentration can be measured by gas chromatography.

Although the pH of the food/beverage article according to the present embodiment is not particularly limited, the pH may be, for example, 3.0 or more, 4.0 or more, or 4.2 or more, and may be 7.0 or less, 6.0 or less, or 5.8 or less, since the savor of the food/beverage article is good.

The food/beverage articles according to the present embodiment can be produced by, for example, adding licorice oily extract containing the compound (I) or the compound (I) to a food/beverage article as a base (preferably liquid seasoning or food) to a concentration of 1 ppm or more, and, if necessary, diluting or concentrating (e.g., deaeration, heating, drying, heating and deaeration at reduced pressure) the resultant and adding various additives, for example.

As confirmed in the following Examples, the compound (I) exhibits antimicrobial action on lactic acid bacteria and/or flat sour bacteria. Therefore, one aspect of the present invention provides an antimicrobial agent to lactic acid bacteria and/or flat sour bacteria containing the compound (I) as an active ingredient.

The antimicrobial agent according to the present embodiment can be used for various harm lactic acid bacteria (lactic acid bacteria encompassed by, for example, Lactobacillus, Streptococcus, and Lactococcus) and flat sour bacteria (for example, Bacillus coagulans).

The antimicrobial agent according to the present embodiment may be any of forms such as a solid (for example, powder), liquid (for example, a solution or a suspension), and a paste. The antimicrobial agent may be any dosage form such as powder, granules, a tablet, a capsule, a solution, a suspension, or a syrup. The above-mentioned various formulations can be prepared by mixing Licoricidin with additives (a vehicle, a binder, a lubricant, a disintegrator, an emulsifier, a surfactant, a base, a solubilizer, a suspending agent, and the like) and molding the resulting mixture, as needed.

The antimicrobial agent according to the present embodiment can be used by adding it to any food/beverage article. Examples of such a food/beverage article include liquid seasonings such as soy sauce, soup stock, Japanese seasoned soup stock, sauce, soup, Worcestershire sauce, dressing, and cooking vinegar; semisolid seasonings such as miso and mayonnaise; pickled Japanese plums; pickles; and delicatessen.

As confirmed in the following Examples, the compound (I) exhibits the action of suppressing the proliferation of lactic acid bacteria and/or flat sour bacteria. Therefore, one aspect of the present invention provides a method for suppressing the proliferation of lactic acid bacteria and/or flat sour bacteria in a food/beverage article, comprising: adjusting the total of the concentrations of the compounds (I) in the food/beverage article to 1 ppm or more.

### EXAMPLES

Hereinafter, the present invention will be described by way of Examples more specifically. However, the present invention is not limited by the following Examples.

### Test Example 1: Antimicrobial Action of Edible Plant Extracts on Lactic Acid Bacteria

### (Preparation of Extract)

To pulverized leaves, stems, or roots of commercially available edible plants (described in Tables 1 and 2) was added sterilized water in the same amount as that of the pulverized material, and the mixture was stirred and mixed with a vortex and left to stand at room temperature for 1 hour. To this mixture was added ethanol in 3 times the volume of the sterilized water, and extraction was performed in an incubator under the conditions of 50° C. and 120 rpm (reciprocal shaking) for 3 hours to obtain 75% ethanol extraction liquid. This extraction liquid was centrifuged at 3000 rpm for 10 minutes to obtain extracts.

### (Preparation of Soy Sauce)

Commercially available low-salt soy sauce (produced by Kikkoman Corporation) was two-fold diluted with sterilized water. The pH of the dilution was adjusted to 4.8 with sodium hydroxide, and the mixture was heat-disinfected to prepare test low-salt soy sauce.

### (Evaluation of Antimicrobial Action)

First, 11.3 mL of test low-salt soy sauce, 0.7 mL of the extract, and lactic acid bacteria (Lactobacillus rennini and Lactobacillus acidlipiscis) were added to a test tube to a final concentration of around 10⁵ cfu/mL, and a SILICOSEN was put in the test tube. The mixture was left to stand at 30° C. for anaerobic culture. The proliferation of lactic acid bacteria produces precipitate of bacterial cells; accordingly, the precipitate produced on the bottom of the test tube was observed over time. The number of days until the generation of the precipitation started in the test, to which the extract was added, was compared with that in a control (mixture to which an aqueous 75% ethanol solution was added instead of the extract), to evaluate the antimicrobial action. The results are shown in Tables 1 and 2.

TABLE 1

| | Difference in |
|---|---|
| | the number of |
| | days until |
| Edible plant | precipitation |
| Kachiwari (pumpkin) | No difference |
| Ogato (pumpkin) | No difference |
| Kamikochi (cucumber) | No difference |
| Bateshirazu (cucumber) | No difference |
| Myoko (tomato) | No difference |
| Heart-heart (tomato) | No difference |
| Chako (cherry tomato) | No difference |
| Brazil cook (cooking tomato) | No difference |
| Shin-etsu mizunasu | No difference |
| Native aonasu | No difference |
| Manganji-amato | No difference |
| Kurokodama-suika | No difference |
| Tenchoha (makuwa) | No difference |
| Chikumano-gosun (carrot) | Difference of |
| | less than 3 days |
| Eruwan (leaf lettuce) | No difference |
| Erugo (butterhead lettuce) | No difference |
| Shimamura kidney bean | No difference |
| Koshigaya kidney bean | No difference |
| Mochitto-corn | No difference |
| Kiso purple turnip | No difference |
| Shinkaiaona (komatsuna) | No difference |
| Yasaiegoma | No difference |
| Fukkura (ruccola) | Difference of |
| | 3 to 6 days |
| Curled mallow | No difference |
| Kawashima kakina | No difference |
| Amaranth | No difference |
| Black-eyed pea | No difference |
| Spontaneous green pepper | No difference |
| Cherry tomato | No difference |
| Miyashige (daikon) | No difference |
| Radish | No difference |
| Beet | No difference |
| Green coin (tatsoi) | No difference |
| Anton (squash) | No difference |
| Y-star (squash) | No difference |
| Celery | Difference of |
| | less than 3 days |
| White Lisbon (long Welsh onion) | No difference |
| Leek (Welsh onion) | No difference |
| Chinese chive | No difference |
| Coriander | No difference |
| Italian parsley | Difference of |
| | less than 3 days |
| Rocket (rucola) | Difference of |
| | less than 3 days |
| Rocky wild (rucola) | Difference of |
| | less than 3 days |
| Sage | No difference |
| Time | No difference |
| Sweet basil | No difference |
| Sweet marjoram | No difference |
| Lemon basil | No difference |
| Lemon balm | No difference |
| Petra (basil) | No difference |
| Bouquet (dill) | Difference of |
| | 7 days or more |
| Nasturtium | Difference of |
| | 3 to 6 days |
| German chamomile | No difference |
| Naga-san (pumpkin) | No difference |
| White cucumber (cucumber) | No difference |
| Spontaneous odama (tomato) | No difference |
| Bonita (tomato) | No difference |
| Broccoli | No difference |
| Red cabbage | No difference |
| White radish sprout | No difference |
| Tsumamina | No difference |
| Turnip | No difference |
| Buckwheat | No difference |
| Water spinach | Difference of |
| | less than 3 days |
| Rocket | Difference of |
| | less than 3 days |
| Basil | No difference |
| Mustard | Difference of |
| | less than 3 days |
| White sesame | No difference |
| Nozawana | No difference |
| Green pea | No difference |
| Black mappe | No difference |
| Alphalpha | No difference |
| Perilla | No difference |
| Endive | No difference |
| Kale | No difference |
| Soup celery | Difference of |
| | 7 days or more |
| Fennel | Difference of |
| | less than 3 days |
| Mike-takana | No difference |
| Green mappe | No difference |
| Bean sprout | No difference |
| Papaya | No difference |
| Koryan-mai mochi | No difference |
| Red rice | No difference |
| Black rice | No difference |
| Linseed | No difference |
| Amaranth | No difference |
| Perilla | No difference |
| Quinoa | No difference |
| Poppy seed | No difference |
| Chicory | Difference of |
| | less than 3 days |
| Winter melon (seed) | Difference of |
| | less than 3 days |
| Pino green | No difference |
| Green spinach | No difference |
| Red beet | No difference |
| Red Asian mustard | Difference of |
| | 3 to 6 days |
| Tatsoi | No difference |
| Choy sum | No difference |
| Aonaga daikon | No difference |
| Koshin daikon | No difference |
| Benimaru daikon | No difference |
| Napa cabbage | No difference |
| Purple-stem mustard | No difference |
| Shiroziku Pak choi | No difference |
| Sweet marjoram | No difference |
| Chervil | No difference |
| Chive | No difference |
| Peppermint | No difference |
| Watercress | No difference |
| Borage | No difference |
| Coriander | No difference |
| Artichoke | No difference |
| Crimson clover | No difference |
| Nigella | No difference |
| Nagaokana | No difference |
| Osakashirona | No difference |
| Hiroshimana | No difference |
| Katsuona | No difference |
| Kumamotokyona | No difference |
| Yamashiona | Difference of |
| | 3 to 6 days |
| Mibuna | No difference |
| Bashona | No difference |
| Kobutakana | Difference of |
| | 3 to 6 days |
| Kalonji/Nigella | No difference |
| Kumquat seed | No difference |
| Bitter melon seed | No difference |
| Pomegranate seed | No difference |
| Prune seed | No difference |
| Coriander | No difference |
| Basil | Difference of |
| | less than 3 days |
| Cayenne pepper | No difference |
| Parsley | No difference |
| Habanero | No difference |
| Paprika | No difference |
| Fenugreek | No difference |
| Fennel | No difference |
| Bhut Jolokia | No difference |
| Fried onion | No difference |
| Marjoram | No difference |
| Onion powder | No difference |
| Red pepper/chili | No difference |
| Lemon grass | Difference of |
| | less than 3 days |
| Kiwi fruit seed extract | No difference |
| Cacao extract | No difference |
| Perilla seed extract | No difference |
| Chinese chive seed extract | No difference |
| Broccoli sprout | No difference |
| Yuzu seed extract | No difference |
| Kanka extract | Difference of |
| | 3 to 6 days |
| Japanese butterbur extra | No difference |
| Buckwheat leaf extract | No difference |
| Satsuma orange extract | No difference |
| Fermented rice germ extract | No difference |
| Black rice extract | No difference |
| Red rice extract | No difference |
| Papaya peel | No difference |
| Dragon fruit peel | No difference |
| Prune peel | No difference |
| Yuzu seed | No difference |
| Karin seed | No difference |
| Sicklefruit fenugreek | No difference |
| Ginger (herb tea) | Difference of |
| | 7 days or more |
| Stevia leaf (herb tea) | Difference of |
| | less than 3 days |

TABLE 2

| | Difference in |
|---|---|
| | the number of |
| | days until |
| Edible plant | precipitation |
| Dandelion leaf (herb tea) | No difference |
| Dandelion root (herb tea) | No difference |
| Peppermint (herb tea) | Difference of |
| | 7 days or more |
| Horsetail (herb tea) | Difference of |
| | less than 3 days |
| Watercress (raw, aboveground portion) | Difference of |
| | less than 3 days |
| Rape (raw, aboveground portion) | No difference |
| Dropwort (raw, aboveground portion) | No difference |
| Nettle (herb tea) | No difference |
| Mallow blue (herb tea) | No difference |
| Yuzu ichimi | Difference of |
| | 3 to 6 days |
| Ginger (powder) | Difference of |
| | 7 days or more |
| Turmeric (powder) | No difference |
| Poppy sheet | No difference |
| Bee pollen | No difference |
| Vanilla bean (whole) | No difference |
| Chinese wolfberry fruit | No difference |
| Gardenia fruit (whole) | No difference |
| Parsley | No difference |
| Udo (raw, aboveground portion) | Difference of |
| | 7 days or more |
| Coriander (raw, whole grass) | Difference of |
| | less than 3 days |
| Fennel (raw, aboveground portion) | Difference of |
| | less than 3 days |
| Sweet basil (freeze-dried) | Difference of |
| | 7 days or more |
| Kaffir lime (whole) | No difference |
| Lemon grass (whole) | No difference |
| Orange flower (herb tea) | No difference |
| Lavender (herb tea) | Difference of |
| | less than 3 days |
| Verbena (herb tea) | No difference |
| Coconut long | No difference |
| Blue poppy seed | No difference |
| Mango powder | Difference of |
| | less than 3 days |
| Ginger powder | Difference of |
| | 7 days or more |
| Wild herb tea jack bean | No difference |
| Wild herb tea field horsetail | No difference |
| Wild herb tea mulberry leaf | Difference of |
| | 3 to 6 days |
| Wild herb tea aloe | No difference |
| Burdock powder | No difference |
| Sprouted brown rice powder | No difference |
| Spinach powder | No difference |
| Carrot powder | No difference |
| Pumpkin powder | No difference |
| Wild herb tea persimmon leaf | No difference |
| Wild herb tea corn | No difference |
| Wild herb tea five-leaf ginseng | No difference |
| Wild herb tea pu-erh tea | No difference |
| Wild herb tea simon tea | No difference |
| Wild herb tea reishi | No difference |
| Wild herb tea loquat leaf | No difference |
| Wild herb tea plantain | No difference |
| Wild herb tea mugwort | No difference |
| Wild herb tea lotus leaf | No difference |
| Wild herb tea Veitch's bamboo | No difference |
| Wild herb tea dokudami | No difference |
| Wild herb tea roasted green tea | No difference |
| Wild herb tea buckwheat tea | No difference |
| Wild herb tea senna tea | No difference |
| Wild herb tea Chinese wolfberry fruit | No difference |
| Komatsuna powder | No difference |
| Lemon powder | No difference |
| Purple sweet potato powder | No difference |
| Lotus root powder | No difference |
| Corn powder | No difference |
| Yuzu powder | No difference |
| Bamboo peel | No difference |
| Hard rush | No difference |
| Cherry leaf, green | No difference |
| Cherry life, brown | No difference |
| Veitch's bamboo, domestic, dark green | No difference |
| Veitch's bamboo, Chinese, green | No difference |
| Okra powder | No difference |
| Edamame powder | No difference |
| Horsetail tea | No difference |
| Tomato powder | No difference |
| Garlic powder | No difference |
| Dandelion coffee | No difference |
| Black soybean flour | No difference |
| Jew's mallow powder | No difference |
| Jerusalem artichoke powder | No difference |
| Kintoki ginger powder | Difference of |
| | 3 to 6 days |
| Green barley powder | No difference |
| lees of linseed oil, undried | No difference |
| Sweet potato powder | No difference |
| Cabbage powder | No difference |
| Yacon leaf powder | Difference of |
| | less than 3 days |
| Red beefsteak plant powder | Difference of |
| | less than 3 days |
| Ashitaba powder | No difference |
| Ginger powder | Difference of |
| | 3 to 6 days |
| Yacon potato powder | No difference |
| Shitake powder | No difference |
| Celery powder | Difference of |
| | 3 to 6 days |
| Wild turmeric powder | Difference of |
| | 7 days or more |
| Cleavers | No difference |
| Hibiscus | Difference of |
| | 3 to 6 days |
| Skull cap | No difference |
| Sweet clover | No difference |
| Spinach | No difference |
| Broccoli | No difference |
| Superior (lettuce) | No difference |
| Swiss chard | No difference |
| California red (paprika) | No difference |
| Red chicory | Difference of |
| | less than 3 days |
| Oresh (endive) | No difference |
| Tokyo bekana (mustard greens) | No difference |
| Green romaine | No difference |
| Red romaine | No difference |
| Green oak | No difference |
| Red oak | No difference |
| Baby purple | No difference |
| Chirimen mustard | Difference of |
| | 3 to 6 days |
| Bok choy | No difference |
| Water spinach | No difference |
| Cardamon | Difference of |
| | 7 days or more |
| Korean red pepper | No difference |
| Caraway | Difference of |
| | 7 days or more |
| Mustard seed/brown | No difference |
| Mustard seed/yellow | No difference |
| Mustard/yellow | No difference |
| Ginkgo (herb tea) | Difference of |
| | 7 days or more |
| Safflower (herb tea) | No difference |
| Rosemary | No difference |
| Bird's eye | Difference of |
| | less than 3 days |
| Kesshoku | No difference |
| Atractylodes rhizome | Difference of |
| | 7 days or more |
| Dong quai | No difference |
| Astragalus root | No difference |
| Byakushi | Difference of |
| | less than 3 days |
| Dried magnolia leaf | No difference |
| Radish | No difference |
| Garland chrysanthemum | No difference |
| Green onion sprout | No difference |
| Japanese honewort | Difference of |
| | 7 days or more |
| Pink radish | No difference |
| Green radish | No difference |
| Vitamin-na | No difference |
| Onion | No difference |
| Ginger mince | Difference of |
| | 7 days or more |
| Tamarind | No difference |
| Tarragon/Estragon | Difference of |
| | 7 days or more |
| Citrus unshiu peel | Difference of |
| | less than 3 days |
| Dill weed | Difference of |
| | less than 3 days |
| Dill seed | Difference of |
| | 7 days or more |
| Dry onion | No difference |
| Nutmeg | Difference of |
| | 3 to 6 days |
| Red jujube | No difference |
| Kinshinsai | No difference |
| Broccoli powder | No difference |
| Lemon peel | Difference of |
| | 7 days or more |
| Japanese pepper | No difference |
| Purple shallot (red onion) | No difference |
| Ashitaba powder | Difference of |
| | less than 3 days |
| Black rice powder | No difference |
| Angelica | No difference |
| German chamomile | Difference of |
| | 3 to 6 days |
| Yam powder | No difference |
| Kale powder | No difference |
| Mulberry leaf powder | No difference |
| Gymnema sylvestre powder | No difference |
| Dried immature orange powder | No difference |
| Licorice powder | Difference of |
| | 7 days or more |

### Test Example 2: Sensory Evaluation

For 19 extracts the antimicrobial action of which was found (the difference from the control was 7 days or more in the number of days until the proliferation) in Test Example 1, whether the extract was suitable as an ingredient in view of fragrance and taste was sensory evaluated using the following evaluation criterion by a trained evaluator. The results are shown in Table 3. In the cell of “portion used” in the table, 1 indicates seed (seed coat) or fruit (fruit coat), 2 indicates leaves, stems, flowers, or trunks, and 3 indicates roots.

### Evaluation Criterion

TABLE 3

| | Portion | | | Overall |
|---|---|---|---|---|
| Material | used | Fragrance | Taste | rating |
| Bouquet (Dill) | 1 | Δ | — | X |
| Japanese honewort | 1 | Δ | — | X |
| Soup celery | 1 | Δ | — | X |
| Ginger mince | 3 | ◯ | Δ | Δ |
| Tarragon/Estragon | 2 | ◯ | Δ | Δ |
| Dill seed | 1 | Δ | — | X |
| Cardamon | 1 | X | — | X |
| Caraway | 1 | Δ | — | X |
| Ginkgo (Herb tea) | 2 | Δ | — | X |
| Ginger (Herb tea) | 3 | ◯ | Δ | Δ |
| Peppermint (Herb tea) | 2 | Δ | — | X |
| Ginger (powder) | 3 | ◯ | Δ | Δ |
| Udo (raw, aboveground portion) | 2 | Δ | — | X |
| Sweet basil (freeze-dried) | 2 | X | — | X |
| Lemon peel | 1 | Δ | — | Δ |
| Ginger powder | 3 | ◯ | Δ | Δ |
| Atractylodes rhizome | 2 | Δ | — | X |
| Wild turmeric powder | 3 | Δ | — | X |
| Licorice powder | 3 | ◯ | ◯ | ◯ |

Evaluation Criterion
◯: Suitable as an ingredient
Δ: Less suitable as an ingredient
X: Absolutely not suitable as an ingredient
—: Not evaluated

It was estimated from Table 3 that the extract of licorice powder was the most suitable as an ingredient in the overall rating. The extract was added to commercially available low-salt soy sauce (produced by Kikkoman Corporation) to 100 ppm and subjected to sensory evaluation in terms of fragrance and taste by a panel consisting of five trained evaluators. As a result, it was confirmed that the flavor of the low-salt soy sauce was not impaired, and that the extract was suitable as an ingredient.

### Test Example 3: Identification of a Substance in Licorice Powder Having Antimicrobial Action on Lactic Acid Bacteria

### (Preparation of Extract)

To 1 kg of licorice powder (NIPPON FUNMATSU YAKUHIN Co., LTD.) was added 5 L of chloroform (KANTO CHEMICAL CO., INC.), and the mixture was stirred at room temperature for 2 hours and then filtered using a folded filter paper (5C). This filtrate was concentrated to dryness using an evaporator to obtain 11 g of an extract.

### (Fraction of Substance Having Antimicrobial Action)

A column (3 L) was filled with silica gel (Disogel IR-60-40/63A Cat. 1002A). Around 11 g of the above-mentioned obtained extract was dissolved in around 30 ml of chloroform, and the mixture was fractionated into 13 fractions (A to M in order of elution) with the following solvents sequentially while monitoring by TLC analysis at a flow rate of 40 ml/minute and at 220 nm.
1) Hexane (6 L)
2) Hexane:ethyl acetate=4:1 (10 L)
3) Hexane:ethyl acetate=3:1 (12 L)
4) Hexane:ethyl acetate=2:1 (10 L)

### (Evaluation of Antimicrobial Action of Fractions)

Each fraction was added to a plate count agar with BCP (Nissui) to 200 μg/mL, 67 μg/mL, 22 μg/mL, or 7 μg/mL. Then, 1 platinum loop of a bacterial suspension of lactic acid bacteria (Lactobacillus rennini and Lactobacillus acidipiscis, 10⁶ to 10⁷ cfu/mL) was applied. The bacteria were subjected to anaerobic culture at 30° C. for 8 days, and the effect of suppressing the proliferation of lactic acid bacteria was evaluated. In the evaluation, the antimicrobial unit of each of the fractions was calculated using the following expression (1), and the ratio of contribution to the antimicrobial action was calculated from the obtained antimicrobial unit of the fraction using the following expression (2). The results are shown in Table 4.

Antimicrobial unit of a fraction 100/concentration at which the fraction exhibits antimicrobial action×fraction volume Expression (1):

Rate of contribution to antimicrobial action (%)=(antimicrobial unit of the fraction/antimicrobial unit of all fractions)×100 Expression (2):

TABLE 4

| | | Active | | |
|---|---|---|---|---|
| | Amount of | concentration | Antimicrobial | Contribution |
| Fraction | fraction (g) | (μg/mL) | unit of fraction | rate (%) |
| A | 0.3 | >200 | — | — |
| B | 1.3 | >200 | — | — |
| C | 0.2 | >200 | — | — |
| D | 0.82 | >200 | — | — |
| E | 0.73 | >200 | — | — |
| F | 0.2 | 22 | 0.91 | 14.5 |
| G | 0.31 | 200 | 0.16 | 2.5 |
| H | 0.21 | 200 | 0.11 | 1.7 |
| I | 0.86 | 22 | 3.91 | 62.2 |
| J | 0.33 | 200 | 0.16 | 2.6 |
| K | 0.41 | 200 | 0.2 | 3.3 |
| L | 0.61 | 200 | 0.31 | 4.9 |
| M | 1.06 | 200 | 0.53 | 8.4 |

As clear from Table 4, it was observed that the fractions F and I each had strong antimicrobial action, and it was strongly estimated that the main antimicrobial component of licorice powder was contained especially in the fraction I. Then, the 13C-NMR spectrum was measured to identify the antimicrobial component contained in the fraction I. Consequently, the antimicrobial component was estimated to be licoricidin, and when the 13C-NMR spectrum of the antimicrobial component was compared with the 13C-NMR spectrum of the preparation (produced by ChemFaces), it was confirmed that both match each other. When the antimicrobial component was subjected to TOF (time-of-flight) mass spectrometry, it was confirmed that the antimicrobial component exhibits the peak of [M+H]⁺ at an m/z of 425, as with licoricidin.

### Test Example 4: Antimicrobial Action of Licoricidin on Lactic Acid Bacteria in Liquid Seasoning (1)

### (1) Production of Liquid Seasonings

### (Production of Soy Sauce)

Commercially available whole soybean soy sauce (produced by Kikkoman Corporation) was electrodialyzed and then concentrated under reduced pressure to obtain soy sauce at a salt concentration of 0% (w/v) and an alcohol concentration of 0% (v/v). Then, salt and/or alcohol was added to the above-mentioned obtained soy sauce, and the mixture was heat-treated to produce soy sauces at salt concentrations and alcohol concentrations shown in the following Table 5. The pHs of the soy sauces were 4.9 to 5.0.

TABLE 5

| Salt concentration | Alcohol concentration |
|---|---|
| (%(w/v)) | (%(v/v)) |
| 0 | 0 |
| | 5 |
| | 10 |
| 4 | 0 |
| | 5 |
| | 10 |
| 8 | 0 |
| | 5 |
| | 10 |
| 12 | 0 |
| | 5 |
| | 10 |

(Production of Bonito Stock)

Bonito stocks was produced using materials shown in the following Table 6. Specifically, components contained in dried bonito was extracted with alcohol and water; sugar, salt, and seasonings were blended with the obtained extract; and the resulting mixture was heated to produce bonito stock. The salt concentration of the obtained bonito stock was 0.2% (w/v), the alcohol concentration was 0.15% (v/v), and the pH was 6.1.

TABLE 6

| Ingredient | Amount blended | |
|---|---|---|
| Dried bonito | 7.5 | g |
| Sugar | 3.2 | g |
| Salt | 2.0 | g |
| Seasonings (amino acids | 4.0 | g |
| and the like) | | |
| Alcohol | 1.5 | mL |
| Water | 993 | mL |
| Total | 1000 | mL |

(Production of Sauce)

Sauce was produced using materials shown in the following Table 7. Specifically, Koikuchi soy sauce (produced by Kikkoman Corporation), granulated sugar, potato starch, and water were blended in amounts shown in following Table 7, and the resulting mixture was heated to produce sauce. The salt concentration of the obtained sauce was 5.3% (w/v), the alcohol concentration was 0.8% (v/v), and the pH was 5.0.

TABLE 7

| Ingredient | Amount blended (g) |
|---|---|
| Koikuchi soy sauce | 354.6 |
| Granulated sugar | 121 |
| Dogtooth violet starch | 20 |
| Water | 504.4 |
| Total | 1000 |

(Production of Japanese Seasoned Soup Stock)

Japanese seasoned soup stock was produced using materials shown in following Table 8. Specifically, the components contained in dried bonito was extracted with water; Koikuchi soy sauce (produced by Kikkoman Corporation), sugar, and salt were blended with the obtained extract; and the resulting mixture was heated to produce Japanese seasoned soup stock. The salt concentration of the obtained Japanese seasoned soup stock was 3% (w/v), the alcohol concentration was 0.4% (v/v), and the pH was 5.5.

TABLE 8

| Ingredient | Amount blended | |
|---|---|---|
| Koikuchi soy sauce | 105 | mL |
| Dried bonito | 31.3 | g |
| Sugar | 40 | g |
| Salt | 13.6 | g |
| Water | 888 | mL |
| Total | 1000 | mL |

(2) Evaluation of Antimicrobial Action

Licoricidin (produced by ChemFaces) was added to each of the liquid seasonings produced in the above-mentioned (1) to concentrations of 1 ppm, 5 ppm, 10 ppm, 25 ppm, and 50 ppm. Lactic acid bacteria described in the following Table 9 were then added to 10⁶ to 10⁷ cells/mL and cultured at 30° C. for 7 days. After the culture, the number of lactic acid bacteria was measured using the GAM agar medium “Nissui”. Liquid seasoning to which licoricidin was not added was used as a control group, and the antimicrobial activity was evaluated in comparison with the number of lactic acid bacteria in the control group. The evaluation was performed using the criterion shown below. The results are shown in Tables 10 to 16.

### (Evaluation Criterion)

A: The bacterial count decreases to less than 10% of the bacterial count of the control group.
B: The bacterial count decreases to 10% or more and less than 50% of the bacterial count of the control group.
C: The bacterial count decreases to 50% or more and less than 100% of the bacterial count of control group.
D: The bacterial count is the same as the bacterial count of the control group.

TABLE 9

| Lactic acid | | Species culture |
|---|---|---|
| bacteria | Species name | collection ID |
| A | Lactobacillus rennini | DSM 20253 |
| B | Lactobacillus rennini | DSM 17732 |
| C | Lactobacillus acidipiscis | NBRC 102163 |
| D | Lactobacillus acidipiscis | NBRC 102164 |

TABLE 10
Soy sauce; Lactic acid bacteria A

| Salt | Alcohol | | | | | |
|---|---|---|---|---|---|---|
| concentration | concentration | Licoricidin concentration (ppm) | | | | |
| (%(w/v)) | (%(v/v)) | 1 | 5 | 10 | 25 | 50 |
| 0 | 0 | D | B | B | A | A |
| | 5 | C | B | B | A | A |
| | 10 | C | C | C | A | A |
| 4 | 0 | B | A | A | A | A |
| | 5 | C | A | A | A | A |
| | 10 | A | A | A | A | A |
| 8 | 0 | C | A | A | A | A |
| | 5 | B | A | A | A | A |
| 12 | 0 | D | A | A | A | A |
| | 5 | A | A | A | A | A |

In soy sauce at a salt concentration of 8% (w/v) or more and an alcohol concentration of 10% (w/v) in control groups, bacteria did not grow. If the Licoricidin concentration was 0.1 ppm, the antimicrobial activity was not found under all the test conditions.

TABLE 11
Soy sauce; Lactic acid bacteria B

| Salt | Alcohol | | | | | |
|---|---|---|---|---|---|---|
| concentration | concentration | Licoricidin concentration (ppm) | | | | |
| (%(w/v)) | (%(v/v)) | 1 | 5 | 10 | 25 | 50 |
| 0 | 0 | B | B | B | A | A |
| 4 | 0 | B | A | A | A | A |
| 8 | 0 | C | A | A | A | A |
| 12 | 0 | A | A | A | A | A |

TABLE 12
Soy sauce; Lactic acid bacteria C

| Salt | Alcohol | | | | | |
|---|---|---|---|---|---|---|
| concentration | concentration | Licoricidin concentration (ppm) | | | | |
| (%(w/v)) | (%(v/v)) | 1 | 5 | 10 | 25 | 50 |
| 0 | 0 | C | C | B | A | A |
| 4 | 0 | C | A | A | A | A |
| 8 | 0 | D | A | A | A | A |
| 12 | 0 | B | A | A | A | A |

TABLE 13
Soy sauce; Lactic acid bacteria D

| Salt | Alcohol | | | | | |
|---|---|---|---|---|---|---|
| concentration | concentration | Licoricidin concentration (ppm) | | | | |
| (%(w/v)) | (%(v/v)) | 1 | 5 | 10 | 25 | 50 |
| 0 | 0 | C | C | B | A | A |
| 4 | 0 | D | A | A | A | A |
| 8 | 0 | C | A | A | A | A |
| 12 | 0 | D | A | A | A | A |

TABLE 14
Bonito stock

| Lactic acid | Licoricidin concentration (ppm) | | | | |
|---|---|---|---|---|---|
| bacteria | 1 | 5 | 10 | 25 | 50 |
| A | C | A | A | A | A |
| B | C | A | A | A | A |
| C | C | A | A | A | A |
| D | A | A | A | A | A |

TABLE 15
Sauce

| Lactic acid | Licoricidin concentration (ppm) | | | | |
|---|---|---|---|---|---|
| bacteria | 1 | 5 | 10 | 25 | 50 |
| A | D | D | A | A | A |
| B | D | A | A | A | A |
| C | B | B | A | A | A |
| D | C | C | A | A | A |

TABLE 16
Japanese seasoned soup stock

| Lactic acid | Licoricidin concentration (ppm) | | | | |
|---|---|---|---|---|---|
| bacteria | 1 | 5 | 10 | 25 | 50 |
| A | D | B | A | A | A |
| B | C | A | A | A | A |
| C | D | A | A | A | A |
| D | D | A | A | A | A |

It was confirmed that licoricidin exhibited antimicrobial activity to various lactic acid bacteria under the conditions of various salt concentration and various alcohol concentrations.

### Test Example 5: Antimicrobial Action of Licoricidin on Lactic Acid Bacteria in Liquid Seasoning (2)

### (Production of Soy Sauce)

Commercially available whole soybean soy sauce (produced by Kikkoman Corporation) was electrodialyzed and then concentrated under reduced pressure to obtain soy sauce at a salt concentration of 0% (w/v) and an alcohol concentration of 0% (v/v). Then, salt and/or alcohol was added to the above-mentioned obtained soy sauce, and hydrochloric acid or an aqueous sodium hydroxide solution was then added to adjust the pH to a desired pH. Germfree purified water was added, and the mixture was then heat-treated to produce soy sauce at the salt concentration, the alcohol concentration, and the pH shown in the following Table 17.

### (Evaluation of Antimicrobial Action)

Licoricidin (produced by ChemFaces) was added to each of the obtained soy sauces to concentrations of 1 ppm, 5 ppm, 10 ppm, 25 ppm, and 50 ppm. Lactic acid bacteria (Lactobacillus rennini (DSM 20253)) were then added to 10⁶ to 10⁷ cells/mL and cultured at 30° C. for 7 days. After the culture, the number of lactic acid bacteria was measured using the GAM agar medium “Nissui”. Soy sauce to which licoricidin was not added was used as a control group, and the antimicrobial activity was evaluated in comparison with the number of lactic acid bacteria in the control group. The evaluation was performed using the same criterion as in Test Example 4. The results are shown in Tables 18 and 19.

TABLE 17

| Salt | Alcohol | |
|---|---|---|
| concentration | concentration | |
| (%(w/v)) | (%(v/v)) | pH |
| 0 | 0 | 4.2, 4.6, 5.0, 5.4, 5.8 |
| | 5 | 4.2, 4.6, 5.0, 5.4, 5.8 |
| 4 | 0 | 4.2, 4.6, 5.0, 5.4, 5.8 |
| | 5 | 4.2, 4.6, 5.0, 5.4, 5.8 |

TABLE 18
Salt concentration 0% (w/v)

| | |
|---|---|
| Alcohol | |
| concentration | Licoricidin concentration (ppm) |

| pH | (%(v/v)) | 1 | 5 | 10 | 25 | 50 |
|---|---|---|---|---|---|---|
| 4.2 | 0 | B | B | A | A | A |
| | 5 | B | A | A | A | A |
| 4.6 | 0 | C | B | A | A | A |
| | 5 | C | A | A | A | A |
| 5.0 | 0 | D | B | B | A | A |
| | 5 | C | B | B | A | A |
| 5.4 | 0 | D | D | C | C | C |
| | 5 | B | A | A | A | A |
| 5.8 | 0 | D | D | D | D | D |
| | 5 | B | A | A | A | A |

TABLE 19
Salt concentration 4% (w/v)

| | |
|---|---|
| Alcohol | |
| concentration | Licoricidin concentration (ppm) |

| ph | (%(v/v)) | 1 | 5 | 10 | 25 | 50 |
|---|---|---|---|---|---|---|
| 4.2 | 0 | B | A | A | A | A |
| | 5 | C | A | A | A | A |
| 4.6 | 0 | D | D | A | A | A |
| | 5 | B | A | A | A | A |
| 5.0 | 0 | B | A | A | A | A |
| | 5 | C | A | A | A | A |
| 5.4 | 0 | B | A | A | A | A |
| | 5 | C | A | A | A | A |
| 5.8 | 0 | D | A | A | A | A |
| | 5 | D | D | A | A | A |

### Test Example 6: Antimicrobial Action of Oxygen-Containing Heterocyclic Compounds on Lactic Acid Bacteria in Liquid Seasoning (1)

### (Production of Soy Sauce)

Commercially available whole soybean soy sauce (produced by Kikkoman Corporation) was electrodialyzed and then concentrated under reduced pressure to produce soy sauce at a salt concentration of 0% (w/v) and an alcohol concentration of 0% (v/v). Then, salt and/or alcohol was added to the above-mentioned obtained soy sauce, and the mixture was then heat-treated to produce soy sauce at a salt concentration of 4% (w/v) and an alcohol concentration of 5% (v/v).

### (Evaluation of Antimicrobial Action)

The test substance described in Table 20 was added to the above-mentioned produced soy sauce to concentrations of 1 to 100 ppm. Lactic acid bacteria (Lactobacillus rennini (DSM 20253)) were then added to 10⁶ to 10⁷ cells/mL and cultured at 30° C. for 7 days. After the culture, the number of lactic acid bacteria was measured using the GAM agar medium “Nissui”. Soy sauce to which licoricidin was not added was used as a control group, and the antimicrobial activity was evaluated in comparison with the number of lactic acid bacteria in the control group. The evaluation was performed using the criterion shown below. The results are shown in Table 20.

### (Evaluation Criterion)

A: The bacterial count decreases to less than 10% of the bacterial count of the control group.
B: The bacterial count decreases to 10% or more and less than 50% of the bacterial count of control group.
C: The bacterial count decreases to 50% or more and less than 100% of the bacterial count of the control group.
D: The bacterial count is the same as the bacterial count of the control group.
-: Not evaluated

TABLE 20

| Test substance | Test substance concentration (ppm) | | | | | |
|---|---|---|---|---|---|---|
| (manufacturer) | 1 | 5 | 10 | 25 | 50 | 100 |
| Licoricidin | D | A | A | A | A | — |
| (ChemFaces) | | | | | | |
| Gancaonin I | C | A | A | A | A | A |
| (ChemFaces) | | | | | | |
| 8-(γ,γ-dimethylallyl)-wighteone | C | A | A | A | A | A |
| (ALB Technology Limited) | | | | | | |
| Glycycoumarin | D | B | B | A | A | A |
| (ChemFaces) | | | | | | |
| Glyasperin C | B | B | B | A | A | A |
| (ChemFaces) | | | | | | |
| Glycyrin | D | B | B | A | A | A |
| (ChemFaces) | | | | | | |
| Isoangustone A | B | A | A | A | A | A |
| (ChemFaces) | | | | | | |
| Licoarylcoumarin | D | C | C | B | B | A |
| (ChemFaces) | | | | | | |

It was confirmed that the oxygen-containing heterocyclic compounds other than licoricidin had antimicrobial activity, as with licoricidin.

### Test Example 7: Antimicrobial Action of Oxygen-Containing Heterocyclic Compounds on Lactic Acid Bacteria in Liquid Seasoning (2)

### (Evaluation of Antimicrobial Action)

The test substance described in Table 21 was added to the soy sauce produced in Test Example 6 to concentrations of 0.1 ppm, 0.5 ppm, 1 ppm, 5 ppm, 25 ppm, and 50 ppm. Lactic acid bacteria (Lactobacillus fructivores (NBRC 13954)) were then added to 10⁴ to 10⁵ cells/mL and cultured at 30° C. for 7 days. After the culture, the number of lactic acid bacteria was measured using Difco Lactobacilli MRS Agar. Soy sauce to which the test substance was not added was used as a control group, and the antimicrobial activity was evaluated in comparison with the number of lactic acid bacteria in the control group. The evaluation was performed using the following criterion. The results are shown in Table 21.

### (Evaluation Criterion)

A: The bacterial count decreases to less than 10% of the bacterial count of a control group.
B: The bacterial count decreases to 10% or more and less than 50% of the bacterial count of the control group.
C: The bacterial count decreases to 50% or more and less than 100% of the bacterial count of the control group.
D: The bacterial count is the same as the bacterial count of the control group.

TABLE 21

| Test substance | Test substance concentration (ppm) | | | | | |
|---|---|---|---|---|---|---|
| (manufacturer) | 0.1 | 0.5 | 1 | 5 | 25 | 50 |
| Licoricidin | B | B | B | B | A | A |
| (ChemFaces) | | | | | | |
| Gancaonin I | B | C | B | C | B | A |
| (ChemFaces) | | | | | | |
| 8-(γ,γ-dimethylallyl)-wighteone | B | B | B | A | A | A |
| (ALB Technology Limited) | | | | | | |
| Glycycoumarin | B | B | B | B | A | A |
| (ChemFaces) | | | | | | |
| Glyasperin C | B | B | B | B | B | A |
| (ChemFaces) | | | | | | |
| Glycyrin | B | B | B | B | B | B |
| (ChemFaces) | | | | | | |
| Isoangustone A | B | B | C | B | A | A |
| (ChemFaces) | | | | | | |
| Licoarylcoumarin | B | B | B | C | B | B |
| (ChemFaces) | | | | | | |

It was confirmed that the oxygen-containing heterocyclic compounds other than licoricidin had antimicrobial activity, as with licoricidin.

### Test Example 8: Antimicrobial Action of Licoricidin on Flat Sour Bacteria in Liquid Seasoning

### (Production of Japanese Seasoned Soup Stock)

Japanese seasoned soup stock was produced by using materials shown in the following Table 22. Specifically, components contained in dried bonito was extracted with water; Koikuchi soy sauce (produced by Kikkoman Corporation), sugar, and salt were blended with the obtained extract; and the resulting mixture was heated to produce Japanese seasoned soup stock. The salt concentration of the obtained Japanese seasoned soup stock was 3% (w/v), the alcohol concentration was 0.4% (v/v), and the pH was 5.2.

TABLE 22

| Ingredient | Amount blended | |
|---|---|---|
| Koikuchi soy sauce | 105 | mL |
| Dried bonito | 31.3 | g |
| Sugar | 40 | g |
| Salt | 13.6 | g |
| Water | 888 | mL |
| Total | 1000 | mL |

(Evaluation of Antimicrobial Action)

Licoricidin (produced by ChemFaces) was added to the produced Japanese seasoned soup stock to concentrations of 1 ppm, 5 ppm, 10 ppm, 25 ppm, and 50 ppm. Flat sour bacteria (Bacillus coagulans (IFO12714)) were then added to 10⁴ to 10⁵ cells/mL and cultured at 45° C. for 7 days. Japanese seasoned soup stock to which licoricidin was not added was used as a control, and the antimicrobial activity was evaluated by comparing the pHs. The evaluation was performed using the following criterion. The results are shown in Table 23.

### (Evaluation Criterion)

A: There is no change in pH as compared with the control.
B: The pH decreases as compared with the control.

TABLE 23

| Licoricidin (ChemFaces) | 0 | | | | | |
|---|---|---|---|---|---|---|
| (ppm) | (Control) | 1 | 5 | 10 | 25 | 50 |
| pH | 4.1 | 4.1 | 4.1 | 5.3 | 5.3 | 5.2 |
| Evaluation | — | B | B | A | A | A |

It was confirmed that licoricidin exhibited antimicrobial activity to flat sour bacteria.

### Test Example 9: Antimicrobial Action of Licoricidin on Lactic Acid Bacteria in Liquid Seasonings and Food (3)

### (1) Production of Liquid Seasonings and Food

### (Production of Liquid Dressing)

Liquid dressing was produced by using materials shown in the following Table 24. Specifically, Koikuchi soy sauce (produced by Kikkoman Corporation), table vinegar, mirin, granulated sugar, kelp stock, and water were blended in amounts shown in the following Table 24, and the resulting mixture was heated to produce liquid dressing. The salt concentration of the obtained liquid dressing was 2.6% (w/v), the alcohol concentration was 0.8% (v/v), the pH was 4.2.

TABLE 24

| Ingredient | Amount blended (g) |
|---|---|
| Koikuchi soy sauce | 18.72 |
| Table vinegar | 2.54 |
| Mirin | 2.9 |
| Granulated sugar | 5 |
| Kelp stock | 16 |
| Water | 54.84 |
| Total | 100 |

(Production of Lightly-Pickled Vegetables)

Seasoning liquid for lightly-pickled vegetables were produced by using materials shown in the following Table 25. Specifically, Usukuchi soy sauce (produced by Kikkoman Corporation), table vinegar, corn syrup, isomerized sugar, salt, granulated sugar, sodium glutamate (MSG), lemon fruit juice, kelp stock, and water were blended in amounts shown in the following Table 25, and the mixture was heated to produce seasoning liquid for lightly-pickled vegetables. Chinese cabbages were fully soaked therein to produce lightly-pickled vegetables. The salt concentration of the obtained seasoning liquid for lightly-pickled vegetables was 3.1% (w/v), the alcohol concentration was 0.01% (v/v), and the pH was 4.4.

TABLE 25

| Ingredient | Amount blended | |
|---|---|---|
| Usukuchi soy sauce | 0.3 | mL |
| Table vinegar | 0.5 | mL |
| Corn syrup | 15 | g |
| Isomerized sugar | 2 | g |
| Salt | 3 | g |
| Granulated sugar | 4 | g |
| MSG | 0.4 | g |
| Lemon fruit juice | 0.68 | g |
| Kelp stock | 3.2 | mL |
| Water | 83.45 | mL |
| Total | 100 | mL |

(Production of Cooking Vinegar)

Cooking vinegar was produced by using materials shown in the following Table 26. Specifically, table vinegar, salt, granulated sugar, lemon fruit juice, sodium glutamate (MSG), and water in amounts shown in following Table 26 were blended, and the mixture was heated to produce cooking vinegar. The salt concentration of the obtained cooking vinegar was 1.5% (w/v), the alcohol concentration was 0% (v/v), and the pH was 3.8.

TABLE 26

| Ingredient | Amount blended | |
|---|---|---|
| Table vinegar | 0.1 | mL |
| Salt | 1.5 | g |
| Granulated sugar | 10 | g |
| Lemon fruit juice | 5.1 | g |
| MSG | 0.4 | g |
| Water | 87.3 | mL |
| Total | 100 | mL |

### (2) Evaluation of Antimicrobial Action

Licoricidin (produced by ChemFaces) was added to each of the above-mentioned produced liquid dressing, lightly-pickled vegetables, and cooking vinegar to concentrations of 1 ppm, 5 ppm, 10 ppm, 25 ppm, and 50 ppm. Lactic acid bacteria (Lactobacillus rennini (DSM 20253)) were then added to 10⁶ to 10⁷ cells/mL. The bacteria were cultured in the liquid dressing and the lightly-pickled vegetables at 30° C. for 7 days, and the bacteria were cultured in the cooking vinegar at 30° C. for 3 days. After the culture, the number of lactic acid bacteria was measured using the GAM agar medium “Nissui”. The liquid dressing, the lightly-pickled vegetables, and the cooking vinegar to which licoricidin was not added were used as respective control groups, and the antimicrobial activity was evaluated in comparison with the number of lactic acid bacteria in the control group. The evaluation was performed using the same criterion as in Test Example 4. The results are shown in Table 27.

### (Evaluation Criterion)

A: The bacterial count decreases to less than 10% of the bacterial count of the control group.
B: The bacterial count decreases to 10% or more and less than 50% of the bacterial count of the control group.
C: The bacterial count decreases to 50% or more and less than 100% of the bacterial count of control group.
D: The bacterial count is the same as the bacterial count of the control group.

TABLE 27

| | Licoricidin concentration (ppm) | | | | |
|---|---|---|---|---|---|
| Evaluation sample | 1 | 5 | 10 | 25 | 50 |
| Liquid dressing | A | A | A | A | A |
| Lightly-pickled vegetables | B | C | A | A | A |
| Cooking vinegar | A | A | A | A | A |

It was confirmed that licoricidin exhibited antimicrobial activity to lactic acid bacteria in the various liquid seasonings and the food.

1. A food/beverage article comprising:
one or more compounds represented by formula (I): 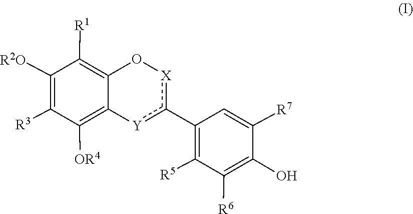
wherein, R¹ and R³ each independently represent a hydrogen atom or a C₂₋₆ alkenyl group, R² and R⁴ each independently represent a hydrogen atom or a C₁₋₄ alkyl group, R⁵ and R⁷ each independently represent a hydrogen atom or a hydroxyl group, R⁶ represents a hydrogen atom or a C₂₋₆ alkenyl group, X represents a direct bond, —CH₂—, —CH═, or —C(═O)—, Y represents —CH₂—, —CH═, or —C(═O)—, and a bond [symbol] represents a single bond or a double bond,
wherein a total of concentrations of the compounds is 1 ppm or more.

2. The food/beverage article according to claim 1, wherein the compound is at least one selected from the group consisting of licoricidin, gancaonin I, 8-(γ,γ-dimethylallyl)-wighteone, glycycoumarin, glyasperin C, glycyrin, isoangustone A, and licoarylcoumarin.

3. The food/beverage article according to claim 1, wherein the food/beverage article is liquid seasoning or food.

4. The food/beverage article according to claim 3, wherein the liquid seasoning is soy sauce, soup stock, Japanese seasoned soup stock, sauce, dressing, or cooking vinegar, and the food is lightly-pickled vegetables.

5-6. (canceled)

7. A method for suppressing proliferation of lactic acid bacteria and/or flat sour bacteria in a food/beverage article, comprising: adjusting a total of concentrations of compounds represented by formula (I) in the food/beverage article to 1 ppm or more: 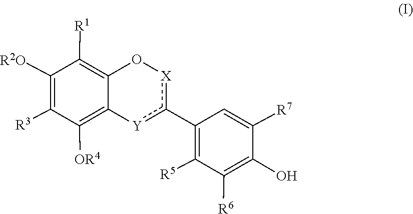
wherein, R¹ and R³ each independently represent a hydrogen atom or a C₂₋₆ alkenyl group, R² and R⁴ each independently represent a hydrogen atom or a C₁₋₄ alkyl group, R⁵ and R⁷ each independently represent a hydrogen atom or a hydroxyl group, R⁶ represents a hydrogen atom or a C₂₋₆ alkenyl group, X represents a direct bond, —CH₂—, —CH═, or —C(═O)—, Y represents —CH₂—, —CH═, or —C(═O)—, and a bond [symbol] represents a single bond or a double bond.

8. The method according to claim 7, wherein the compound is licoricidin, gancaonin I, 8-(γ,γ-dimethylallyl)-wighteone, glycycoumarin, glyasperin C, glycyrin, isoangustone A, or licoarylcoumarin.
